(19) United States
(12) Patent Application Publication
(10) Pub. No.: US 2023/0255766 A1
(43) Pub. Date: Aug. 17, 2023

(54) SHEATH WITH COLLAPSIBLE CAPSULE

(71) Applicant: BOSTON SCIENTIFIC SCIMED, INC., MAPLE GROVE, MN (US)

(72) Inventors: Declan Loughnane, Galway (IE); Tim O'Connor, Galway (IE); Joseph Murphy, Galway (IE)

(73) Assignee: BOSTON SCIENTIFIC SCIMED, INC., MAPLE GROVE, MN (US)

(21) Appl. No.: 18/108,112

(22) Filed: Feb. 10, 2023

Related U.S. Application Data
(60) Provisional application No. 63/309,184, filed on Feb. 11, 2022.

(51) Int. Cl. A61F 2/24

(52) U.S. Cl. CPC A61F 2/2436; A61F 2/2427

(57) ABSTRACT

An outer sheath for use with a medical device delivery system includes a tubular member defined by a circumferential wall and having a distal end, a proximal end, and a lumen extending therebetween along a central longitudinal axis of the tubular member. The tubular has a distal region that is configured to shift between an enlarged configuration and a collapsed configuration. The distal region also includes at least one compression element configured to automatically radially collapse the distal region when the lumen of the distal region is empty.

### CROSS-REFERENCE TO RELATED APPLICATIONS

This application claims the benefit of priority of U.S. Provisional Application No. 63/309,184 filed Feb. 11, 2022, the entire disclosure of which is hereby incorporated by reference.

### TECHNICAL FIELD

The disclosure pertains to medical devices and more particularly a radially collapsible sheath and/or methods of use and/or manufacture thereof.

### BACKGROUND

A wide variety of medical devices have been developed for medical use including, for example, surgical and/or intravascular use. In some instances, performing percutaneous medical procedures may require insertion and/or maneuvering of relatively large medical devices through the vasculature. Such procedures often involve delivering the medical devices through an introducer sheath, which may reduce the potential vessel trauma resulting from forces applied to the vessel wall by the medical device. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example outer sheath includes a tubular member defined by a circumferential wall and having a distal end, a proximal end, and a lumen extending therebetween along a central longitudinal axis of the tubular member, the tubular member having a distal region being configured to shift between an enlarged configuration and a collapsed configuration, and at least one compression element disposed in the distal region, the at least one compression element configured to automatically radially collapse the distal region when the lumen of the distal region is empty.

Alternatively or additionally to the embodiment above, the at least one compression element is embedded within the circumferential wall.

Alternatively or additionally to any of the embodiments above, the at least one compression element includes a plurality of compression elements that are oriented longitudinally.

Alternatively or additionally to any of the embodiments above, the plurality of compression elements includes axial reinforcement fibers embedded within the tubular member.

Alternatively or additionally to any of the embodiments above, at least some of the axial reinforcement fibers have different lengths, such that distal ends of the axial reinforcement fibers are positioned at a common distance from the distal end of the tubular member, and proximal ends of at least some of the axial reinforcement fibers are positioned at different distances from the distal end of the tubular member.

Alternatively or additionally to any of the embodiments above, the axial reinforcement fibers are a plurality of wires.

Alternatively or additionally to any of the embodiments above, the plurality of wires is made of shape-memory material.

Alternatively or additionally to any of the embodiments above, the outer sheath further comprises coupling members coupling each of the plurality of compression elements to adjacent compression elements.

Alternatively or additionally to any of the embodiments above, the coupling members extend circumferentially between adjacent compression elements.

Alternatively or additionally to any of the embodiments above, the coupling members are sutures.

Alternatively or additionally to any of the embodiments above, a first outer diameter of the distal region in the enlarged configuration is larger than a second outer diameter of a remainder of the tubular member.

Alternatively or additionally to any of the embodiments above, the at least one compression element includes a plurality of compression elements that are oriented circumferentially around the tubular member.

Alternatively or additionally to any of the embodiments above, the plurality of compression elements is spaced apart axially.

Alternatively or additionally to any of the embodiments above, the at least one compression element is a single compression element extending helically around the tubular member.

Another example outer sheath comprises a tubular member defined by a circumferential wall and having a distal end, a proximal end, and a lumen extending therebetween along a central longitudinal axis of the tubular member, the tubular member having a distal region being configured to shift between an enlarged configuration and a collapsed configuration, and at least one compression element embedded within the circumferential wall in at least the distal region, the at least one compression element configured to radially collapse the distal region when the lumen of the distal region is empty.

Alternatively or additionally to the embodiment above, the at least one compression element is configured to automatically collapse the distal region when the lumen is empty.

Alternatively or additionally to any of the embodiments above, the at least one compression element is made of shape-memory material.

Alternatively or additionally to any of the embodiments above, the at least one compression element extends helically around the tubular member.

Alternatively or additionally to any of the embodiments above, the at least one compression element extends along an entirety of the distal region.

An example medical device system comprises a replacement heart valve delivery system, and an outer sheath comprising a tubular member defined by a circumferential wall and having a distal end, a proximal end, and a lumen extending therebetween along a central longitudinal axis of the tubular member, the tubular member having a distal region being configured to shift between an enlarged configuration to a collapsed configuration, and a plurality of compression elements disposed in the distal region, the plurality of compression elements configured to automatically radially collapse the distal region when the lumen of the distal region is empty, wherein the replacement heart valve delivery system is configured to slide within the lumen of the tubular member.

Alternatively or additionally to the embodiment above, a distal section of the replacement heart valve delivery system includes a replacement heart valve implant disposed therein.

Alternatively or additionally to any of the embodiments above, when in the enlarged configuration, the distal region of the tubular member is configured to extend over the distal section of the replacement heart valve delivery system containing the replacement heart valve implant.

Alternatively or additionally to any of the embodiments above, a first outer diameter of the distal region of the tubular member when extended over the distal section of the replacement heart valve delivery system containing the replacement heart valve implant, is larger than a second outer diameter of a remainder of the outer sheath.

Alternatively or additionally to any of the embodiments above, when the replacement heart valve delivery system is moved distally out of the outer sheath and the replacement heart valve implant is deployed, the plurality of compression elements is configured to radially collapse the distal region of the tubular member.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIGS. 1, 2, 3, 4A, and 4B are side views of various example outer sheaths of the present application;

FIGS. 5A-5C illustrate steps of delivering a replacement heart valve from an example compressible outer sheath;

FIG. 6 is a cross sectional view of an example replacement heart valve delivery system; and

FIGS. 7A and 7B are side and perspective views, respectively, of an example compressible nosecone.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single piece, structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are medical devices that may be used within a portion of the cardiovascular system in order to diagnose, treat, and/or repair the system. At least some of the medical devices disclosed herein may include a replacement heart valve (e.g., a replacement aortic valve, a replacement mitral valve, etc.) and may reduce, treat, and/or prevent the occurrence of defects such as (but not limited to) regurgitation, leaflet prolapse, and/or valve stenosis. In addition, the devices disclosed herein may deliver the replacement heart valve percutaneously and, thus, may be much less invasive to the patient, although other surgical methods and approaches may also be used. The devices disclosed herein may also provide a number of additional desirable features and benefits as described in more detail below.

One such treatment procedure is the transcatheter aortic valve replacement (TAVR), also known as transcatheter aortic valve implantation (TAVI). The largest part of the delivery catheter for a TAVR/TAVI valve delivery system is the capsule where the sheathed valve is contained. Though the catheter is smaller proximal to the capsule, it is the outer diameter (OD) of the distal capsule that drives the size of the outer sheath. The outer sheath is generally expandable to allow it to be as low profile as possible but the proximal end (near the hemostasis valve) is generally a fixed diameter and is thus larger than the capsule. As the capsule passes through the expandable section it is also pushed out to a larger OD also, albeit briefly. The expandable section may have issues such as seams not opening and seams leaving sharp edges which can damage the vasculature during removal.

One way to combat these issues and reduce the profile as much as possible is to use an in-line sheath. This is a sheath which is part of the delivery system and sits proximal to the capsule. As such it has the same OD as, or lower than, the capsule. However, when the user is finished with the TAVR delivery system they must also remove the in-line introducer sheath and replace it with an additional introducer sheath to perform any additional tasks such as placing a second pigtail for gradient measurement or balloon post dilatation of the valve. This replacement of the sheath is not only a nuisance factor but it increases procedure time and also increases the opportunity for trauma on the femoral and iliac arteries which could lead to bleeding events or embolizing calcium deposits.

The below described outer sheaths include a collapsible distal capsule that allows for the use of an in-line low profile sheath which can remain in place while the TAVR delivery system is removed. The in-line low profile sheath may then be used for additional post-TAVR procedures.

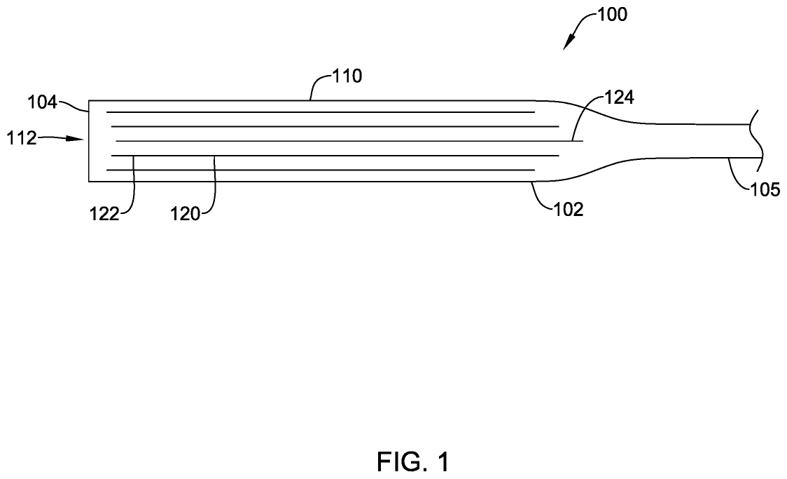

As will be described in greater detail below, FIG. 1 illustrates an example outer sheath 100 which may be utilized to access the vasculature and deliver a medical device. The outer sheath 100 may include a tubular member 102 defined by a circumferential wall, the tubular member 102 having a proximal region 105, a distal region 110, and a lumen 112 extending therebetween along a central longitudinal axis of the tubular member 102. The proximal region 105 may extend proximally from the proximal end of the distal region 110 to a proximal end of the outer sheath 100. The distal region 110 may be configured to shift between an enlarged configuration when a medical device is present in the lumen, as shown in FIG. 1, and a radially collapsed configuration. The outer diameter of the distal region 110 in the enlarged configuration is larger than the outer diameter of the remainder of the tubular member 102. In some embodiments, the outer diameter of the proximal region 105 may be 4.0 mm to 6.0 mm, for example 4.8 mm. The outer diameter of the distal region 110 may be 5.0 mm to 7.0 mm, for example 6.0 mm. It will be understood that the dimensions described in association with the particular embodiments are illustrative only, and that other dimensions of sheaths are contemplated. The distal region 110 may define a collapsible and compressible capsule configured to receive a medical device such as a replacement heart valve. The outer sheath 100 may also include at least one compression element 120 disposed in the distal region 110, where the compression element 120 is configured to radially collapse the distal region 110 when the lumen of the distal region is empty. The term “empty” is understood to mean the lumen is devoid of any other structure, including an implant or portion of a delivery shaft. In some embodiments, the distal region 110 includes a plurality of compression elements 120, as illustrated in FIG. 1. The compression elements 120 may be configured to automatically collapse the distal region 110. In this way, the distal region 110 may be biased in a radially collapsed configuration. This structure of the compression elements 120 automatically collapsing the distal region 110 of the outer sheath provides the advantage of having the distal region 110 collapse as the replacement heart valve or other medical device is moved distally out of the outer sheath 100, and not requiring any manual actuation of a compression element. The compression elements 120 may be configured to collapse the distal region 110 such that the distal region 110 has substantially the same outer diameter as the proximal region 105 when the distal region 110 is empty. In some embodiments, the compression element(s) may be embedded within the wall, such that the compression element 120 is completely covered by the wall.

In the embodiment illustrated in FIG. 1, the distal region 110 of the outer sheath 100 includes a plurality of compression elements 120 that are oriented longitudinally. The plurality of compression elements 120 may be axial reinforcement fibers extending along the length of the distal region 110. The compression elements 120 have radial strength to collapse the distal region 110 but do not need axial strength. The distal region 110 will generally be pulled distally over the compressed medical device, so column strength is not provided by the compression elements 120. In some embodiments, the reinforcement fibers may be embedded within the wall of the tubular member such that they are completely covered by the wall. In some embodiments, at least a portion of some or all of the compression elements 120 may be exposed internally or externally of the distal region 110. At least some of the axial reinforcement fibers 120 may have different lengths, such that when distal ends 122 of the fibers 120 are positioned at a common distance from the distal end 104 of the tubular member, then the proximal ends 124 of at least some of the fibers 120 are positioned at different distances from the distal end 104 of the tubular member 102. This creates staggered proximal ends 124 of the plurality of compression elements 120 which may aid in collapsing the proximal end of the distal region 110, and compressing the distal region 110 as it is withdrawn proximally into an in-line sheath (not shown). Some or all of the plurality of compression elements 120 may extend along the entirety of the distal region 110.

In some embodiments, the compression elements 120 may be wires, and in other embodiments, they may be polymer fibers. In both embodiments, the compression elements 120 may be made of a shape-memory material. When the compression elements 120 are wires, they may be made of stainless steel, nitinol or other metals or stiffer polymer than the distal region 110. Additionally, the outer sheath 100, particularly the proximal region 105, may include metal or polymer braided or woven reinforcement structures.

The distal region 110 may be strong in axial compression during the loading stage as the outer sheath 100 is advanced over a delivery shaft holding a compressed medical device such as a replacement heart valve. The distal region 110 exhibits resistance to radial expansion beyond the enlarged configuration, in order to constrain the valve, and also exhibits tensile strength. There is little need for a resistance to radial compression or crush resistance in the distal region 110, especially once the valve has been deployed, because the lumen of the distal region 110 is empty. The compression elements 120 provide axial reinforcement and also provide the needed axial compression strength during loading the valve while the circular shape of the distal region 110 and polymer construction may provide any needed resistance to radial expansion beyond the enlarged configuration, and enough tensile strength to allow unsheathing.

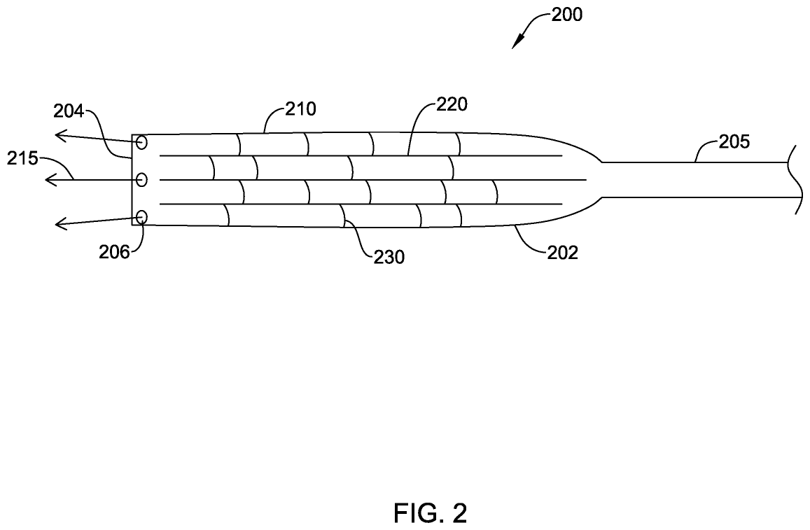

FIG. 2 shows an embodiment of an outer sheath 200 similar to the outer sheath 100 described above, including a tubular member 202 defined by a circumferential wall, the tubular member 202 having a proximal region 205, a distal region 210, and a lumen extending therebetween. Similar to the distal region 110 described above, the distal region 210 of the outer sheath 200 may be configured to shift between an enlarged configuration when a medical device is present in the lumen, as shown in FIG. 2, and a radially collapsed configuration. The distal region 210 may define a collapsible and compressible capsule configured to receive a medical device such as a replacement heart valve. The outer sheath 200 may also include at least one compression element 220 disposed in the distal region 210, where the compression element 220 is configured to radially collapse the distal region 210 when the lumen of the distal region is empty. The outer sheath 200 shown in FIG. 2 differs from that shown in FIG. 1 in that the axial compression elements 220 are attached to each other by coupling members 230. The coupling members 230 may extend circumferentially between adjacent compression elements 220. The coupling members 230 may be sutures, ribbons, cables, or wires embedded within the wall of the tubular member 202 that have higher tensile strength than the polymer forming the tubular member 202, but are not resistant to radial compression, so the coupling members 230 do not interfere with the compression elements 220 collapsing the distal region 210. In some embodiments, the coupling members 230 may provide radial compression, thereby aiding the compression elements 220 in collapsing the distal region 210. The coupling members 230 may couple each compression element 220 to the circumferentially adjacent compression elements. A plurality of coupling members 230 may couple each compression element 220 to its neighbors, and the coupling members 230 may be spaced apart axially, as shown in FIG. 2. As with the compression elements 120 described above, the compression elements 220 may be configured to automatically collapse the distal region 210 of the outer sheath 200 such that the outer diameter of the distal region 210 is substantially the same as the outer diameter of the proximal region 205 when the distal region 210 is empty.

In some embodiments the tubular member 202 may include one or more openings 206 adjacent the distal end 204 of the tubular member 202. The openings 206 may extend completely through the circumferential wall and may be configured to receive sutures or other pulling members that may be used to pull the distal region 210 distally (indicated by arrows 215) over the compressed valve within a valve delivery sheath. It will be understood that openings 206 may be included in any of the outer sheaths described herein. Alternatively, the distal end of the distal region 210 may be devoid of openings and the edge of the distal region 210 may be grasped and pulled over the compressed valve.

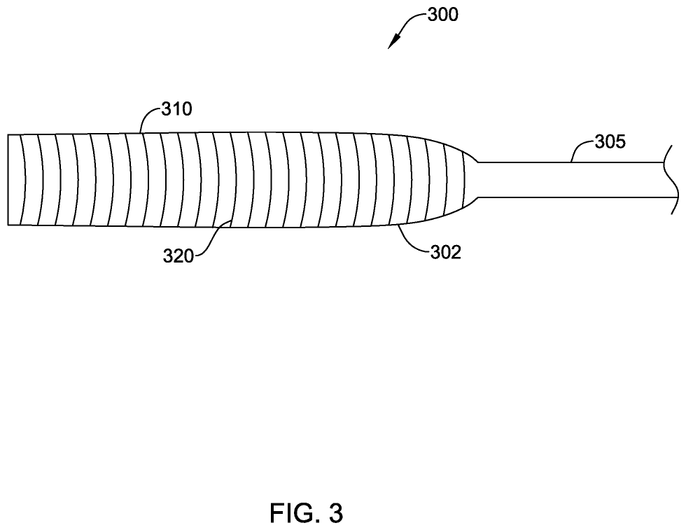

FIG. 3 shows an embodiment of an outer sheath 300 similar to the outer sheath 100 described above, including a tubular member 302 defined by a circumferential wall, the tubular member 302 having a proximal region 305, a distal region 310, and a lumen extending therebetween. Similar to the distal region 110 described above, the distal region 310 of the outer sheath 300 may be configured to shift between an enlarged configuration when a medical device is present in the lumen, as shown in FIG. 3, and a radially collapsed configuration. The distal region 310 may define a collapsible and compressible capsule configured to receive a medical device such as a replacement heart valve. The outer sheath 300 may also include a plurality of compression elements 320 disposed in the distal region 310, where the compression elements 320 are configured to radially collapse the distal region 310 when the lumen of the distal region is empty. The outer sheath 300 shown in FIG. 3 differs from that shown in FIG. 1 in that the plurality of compression elements 320 are oriented circumferentially around the tubular member 302 in the distal region 310. The plurality of compression elements 320 may be spaced apart axially, as shown in FIG. 3, and they may extend along the entire length of the distal region 310. As with the compression elements 120, 220 described above, the compression element 320 may be configured to automatically collapse the distal region 310 of the outer sheath 300 such that the outer diameter of the distal region 310 is substantially the same as the outer diameter of the proximal region 305 when the distal region 310 is empty.

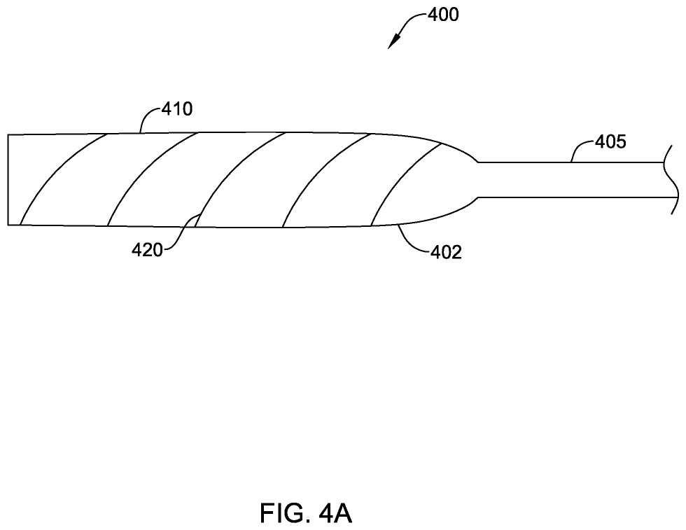

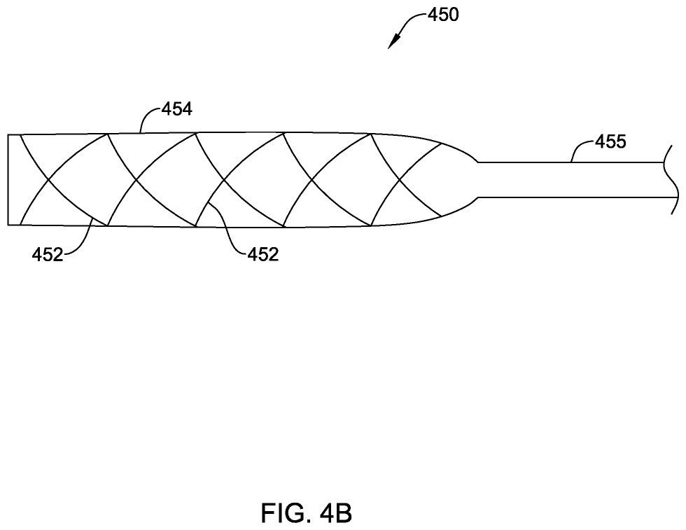

FIG. 4A shows an embodiment of an outer sheath 400 similar to the outer sheath 100 described above, including a tubular member 402 defined by a circumferential wall, the tubular member 402 having a proximal region 405, a distal region 410, and a lumen extending therebetween. Similar to the distal region 110 described above, the distal region 410 of the outer sheath 400 may be configured to shift between an enlarged configuration when a medical device is present in the lumen, as shown in FIG. 4, and a radially collapsed configuration. The distal region 410 may define a collapsible and compressible capsule configured to receive a medical device such as a replacement heart valve. The outer sheath 400 may also include a compression element 420 disposed in the distal region 410, where the compression element 420 is configured to radially collapse the distal region 410 when the lumen of the distal region is empty. The outer sheath 400 shown in FIG. 4 differs from that shown in FIG. 1 in that a single compression element 420 extends helically around the tubular member 402 in the distal region 410. The compression element 420 may extend over the entire length of the distal region 410. As with the compression elements 120, 220, 320 described above, the helical compression element 420 may be configured to automatically collapse the distal region 410 of the outer sheath 400 such that the outer diameter of the distal region 410 is substantially the same as the outer diameter of the proximal region 405 when the distal region 410 is empty.

In another embodiment, shown in FIG. 4B, the outer sheath 450 may include two or more compression elements 452 extending helically over the distal region 454. When a plurality of helical compression elements 452 are present, they may extend in opposite directions, as shown in FIG. 4B. Alternatively, multiple helical compression elements 452 may extend in the same direction. The plurality of compression elements 452 may be spaced apart from one another. As with the compression elements 120, 220, 320, 420 described above, the helical compression elements 452 may be configured to automatically collapse the distal region 454 of the outer sheath 450 such that the outer diameter of the distal region 454 is substantially the same as the outer diameter of the proximal region 455 when the distal region 454 is empty.

In any of the above embodiments, the distal region 110, 210, 310, 410, 454 has minimal axial column strength because it is configured to be pulled distally over the compressed medical device, such as a replacement heart valve, during the loading procedure. The minimal axial column strength may aid in collapsing the distal region after the valve is deployed and the distal region is withdrawn proximally through an in-line sheath.

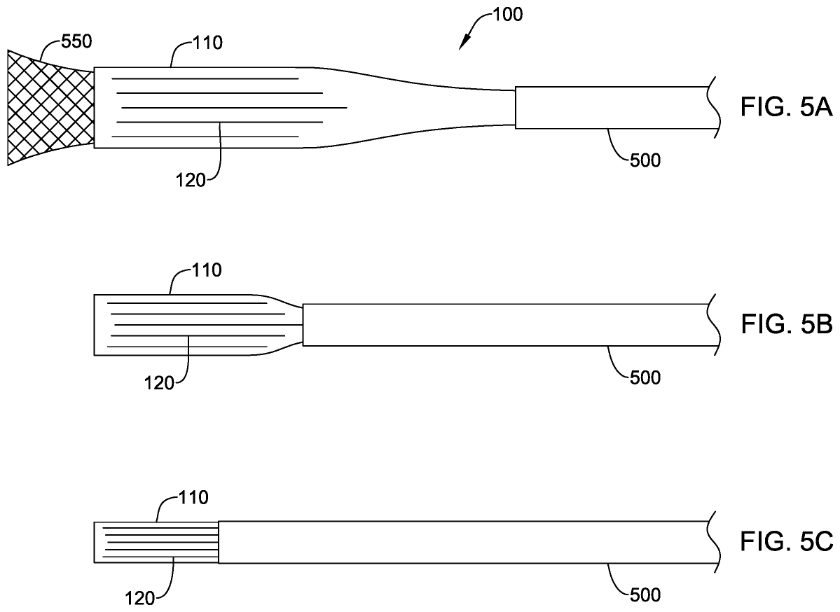

The collapsing action of the distal region 110 of the outer sheath 100 as a medical device is deployed from the lumen is illustrated in FIGS. 5A-5C. In FIG. 5A, the distal region 110 is in the enlarged configuration as a medical device implant 550 is deployed from within the lumen of the distal region 110. The plurality of compression elements 120 are spaced apart circumferentially in the enlarged configuration. An in-line introducer sheath 500 is positioned proximal of the enlarged distal region 110. After the medical device implant 550 has exited the outer sheath 100, the distal region 110 collapses, as indicated by the reduced spacing between the compression elements 120 and the reduced outer diameter of the distal region 110. See FIG. 5B. The outer sheath 100 may then be withdrawn proximally into the in-line introducer sheath 500, which may further collapse the distal region 110 as it enters the in-line sheath, as shown in FIG. 5C.

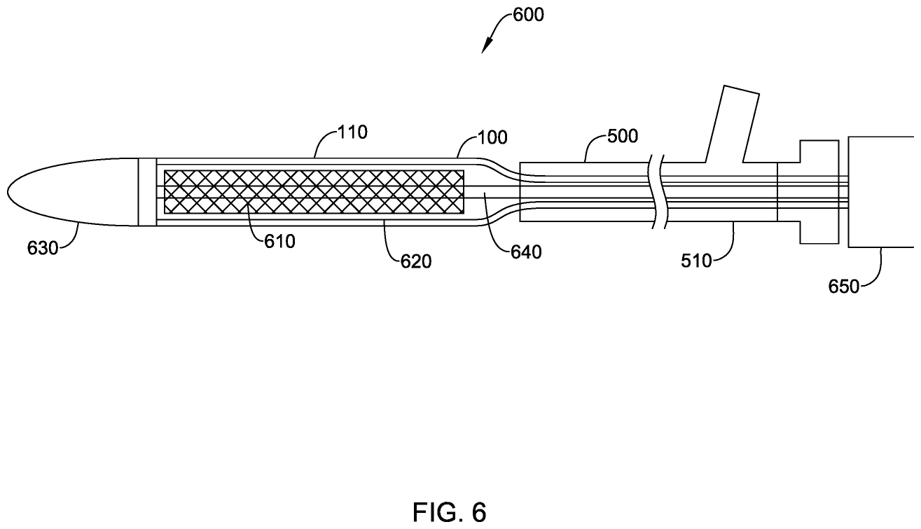

Any of the outer sheaths 100, 200, 300, 400, 450 described above may be used in a medical device system 600 such as the replacement heart valve delivery system shown in FIG. 6. Numerous configurations of replacement heart valve delivery systems are contemplated for use with the outer sheath 100. The exemplary replacement heart valve delivery system 600 includes a delivery sheath 620 having a lumen extending therethrough, and an elongated inner member 640 disposed within the lumen of the delivery sheath 620, wherein the inner member 640 and the delivery sheath 620 are longitudinally slidable relative to each other. A replacement heart valve implant 610 may be disposed within a distal portion of the lumen of the delivery sheath 620 in a collapsed delivery configuration. The replacement heart valve implant 610 may be configured to expand to a deployed configuration when unconstrained by the delivery sheath 620 and/or when actuated to the deployed configuration after releasing the replacement heart valve implant 610 from the delivery sheath 620. The replacement heart valve delivery system 600 may include a handle 650 attached to a proximal end of the delivery sheath 620 and/or the inner member 640. The handle 650 may be operatively connected to the replacement heart valve implant 610 and/or may be configured to actuate the replacement heart valve implant 610 between the collapsed delivery configuration and the deployed configuration. Other configurations are also contemplated, and other medical devices may also be used in connection with the outer sheath 100.

The outer sheath 100 is shown with the distal region 110 in the enlarged configuration disposed over the compressed replacement heart valve implant 610 located within the delivery sheath 620. The compression elements 120 in the distal region 110 are not shown for clarity, however it will be understood that the outer sheath 100 may include any of the compression elements 120, 220, 320, 420, 452 described above. The compression elements 120 may aid in compressing the replacement heart valve implant 610. The delivery sheath 620 may include a nosecone 630 disposed at the distal end thereof and fixed to the inner member 640. The replacement heart valve delivery system may be configured to slide within the lumen of the outer sheath 100. An in-line introducer sheath 500 may extend proximally from a position proximal of the enlarged distal region 110 of the outer sheath.

As shown in FIG. 6, a first outer diameter of the distal region 110 of the outer sheath 100 when extended over the distal section of the replacement heart valve delivery system containing the heart valve implant 610, is larger than a second outer diameter of a remainder of the outer sheath 100, which extends inside the in-line introducer sheath 500. When the replacement heart valve delivery system is moved distally out of the outer sheath 100 and the replacement heart valve implant 610 is deployed, the plurality of compression elements is configured to radially collapse the distal region 110 such that the first outer diameter is substantially the same as the second outer diameter, as shown in FIG. 5C, allowing the outer sheath 100 to be withdrawn proximally through the in-line introducer sheath 500.

The outer sheath 100 may be provided already disposed over the replacement heart valve delivery system, as shown in FIG. 6. Additionally, the in-line introducer sheath 500 including a hemostasis valve 510 may be pre-loaded onto the outer sheath 100. This entire assembly may be provided as a single pre-loaded unit, providing the advantage of having the replacement heart valve implant 610 not have to be pushed through the hemostasis valve 510, thereby avoiding any risk of damage to the replacement heart valve implant or kinking of the delivery system during tracking through the hemostasis valve. Once the replacement heart valve implant 610 has been delivered, the entire system, including the outer sheath 100 may be collapsed and withdrawn proximally through the in-line introducer sheath 500, which may be maintained for use with additional instruments and procedures, such as pressure measurements or angiograms.

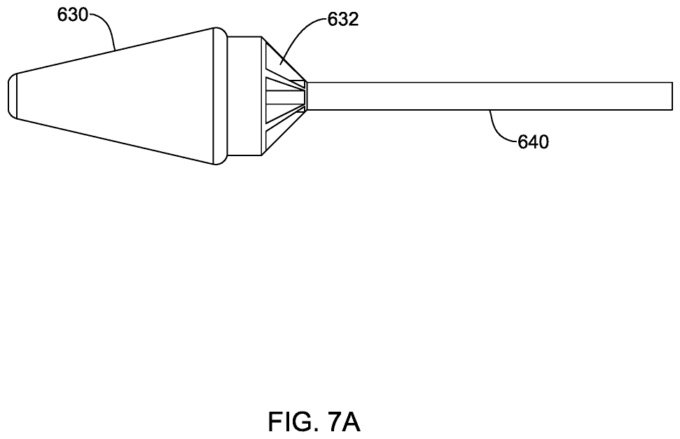

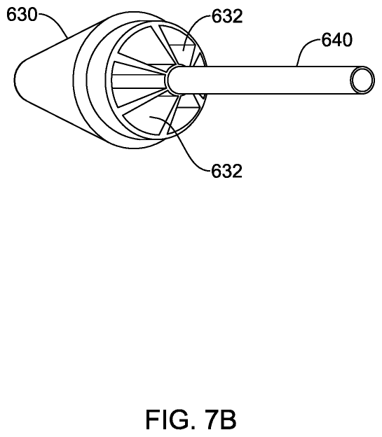

In some embodiments, the nosecone 630 may be compressible. FIGS. 7A and 7B illustrate an example compressible nosecone 630 fixed to the inner member 640. The nosecone 630 may be made of a compressible material such as a polymer, and additionally may include a plurality of voids 632 extending distally from the proximal end of the nosecone 630 towards the distal end of the nosecone 630. The voids 632 may allow the nosecone 630 to be compressed or collapsed as it is withdrawn proximally through the in-line introducer sheath 500.

For simplicity purposes, the following discussion of materials refers to the outer sheath 100 and its compression elements 120. However, this is not intended to limit the devices and methods described herein, as the discussion may be applied to the outer sheaths 200, 300, 400, 450 and their compression elements 220, 320, 420, 452, other elements, members, components, or devices disclosed herein, such as, but not limited to, the delivery sheath 620, the inner member 640, the replacement heart valve implant 610, the nosecone 630, the handle 650, etc. and/or elements or components thereof.

The outer sheath 100 may be made of polymer, elastomeric polyamides, block polyamide/ethers, polyether block amide (PEBA, for example available under the trade name PEBAX®), ethylene tetrafluoroethylene (ETFE), or other polymers generally used in medical catheters. In some embodiments, the outer sheath 100 may be made of a polymer with a hardness of 50-75 shore D. The distal region 110 may be made of a polymer in the higher end of the range while the proximal region 105 may have a hardness in the lower end of the range. Additional examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

The compression elements 120 may be made of a metal, metal alloy, polymer (some examples of which are disclosed above), a metal-polymer composite, ceramics, textiles, combinations thereof, and the like, or other suitable material. Some examples of suitable metals and metal alloys include stainless steel, such as 444V, 444L, 314LV, 304V, or 316 grade stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material. In other embodiments, the compression elements 120 may be made of a shape memory material, such as nitinol or a shape memory polymer. In further embodiments, the compression elements 120 may be made of Kevlar® fibers or polyethylene fibers.

Suitable examples of textile materials for forming the compression elements 120 may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

The entire outer sheath 100, including the expandable distal region 110, may be formed from a single monolithic piece. In other examples, the expandable distal region 110 may be formed from a material different from the proximal region 105, with the two regions attached during manufacture.

Further, it is contemplated that the inner surface and/or outer surface of the outer sheath 100 may include one or more layers and/or coatings, such as a lubricious coating, a hydrophilic coating, a hydrophobic coating, or other suitable coatings, and the like, or may include a lubricant disposed thereon. Some suitable but non-limiting examples of layers and/or coatings are described below.

In at least some embodiments, portions or all of the outer sheath 100, delivery shaft 620, inner shaft 640, and nosecone 630, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the outer sheath 100, particularly the distal region 110. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the outer sheath 100, particularly the distal region 110, to achieve the same result.

In some embodiments, a degree of Magnetic Resonance Imaging (MM) compatibility may be imparted into the outer sheath 100, delivery shaft 620, inner shaft 640, and/or nosecone 630. For example, the outer sheath 100, particularly the distal region 110, may be made of a material that does not substantially distort the image and create substantial artifacts (e.g., gaps in the image). Certain ferromagnetic materials, for example, may not be suitable because they may create artifacts in an MM image. The outer sheath 100, particularly the distal region 110 may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An outer sheath, comprising:
a tubular member defined by a circumferential wall and having a distal end, a proximal end, and a lumen extending therebetween along a central longitudinal axis of the tubular member, the tubular member having a distal region being configured to shift between an enlarged configuration and a collapsed configuration; and
at least one compression element disposed in the distal region, the at least one compression element configured to automatically radially collapse the distal region when the lumen of the distal region is empty.

2. The outer sheath of claim 1, wherein the at least one compression element includes a plurality of compression elements that are oriented longitudinally.

3. The outer sheath of claim 2, wherein the plurality of compression elements includes axial reinforcement fibers embedded within the tubular member.

4. The outer sheath of claim 3, wherein at least some of the axial reinforcement fibers have different lengths, such that distal ends of the axial reinforcement fibers are positioned at a common distance from the distal end of the tubular member, and proximal ends of at least some of the axial reinforcement fibers are positioned at different distances from the distal end of the tubular member.

5. The outer sheath of claim 3, wherein the axial reinforcement fibers are a plurality of wires.

6. The outer sheath of claim 2, further comprising coupling members coupling each of the plurality of compression elements to adjacent compression elements.

7. The outer sheath of claim 6, wherein the coupling members extend circumferentially between adjacent compression elements.

8. The outer sheath of claim 6, wherein the coupling members are sutures.

9. The outer sheath of claim 1, wherein a first outer diameter of the distal region in the enlarged configuration is larger than a second outer diameter of a remainder of the tubular member.

10. The outer sheath of claim 1, wherein the at least one compression element includes a plurality of compression elements that are oriented circumferentially around the tubular member.

11. The outer sheath of claim 10, wherein the plurality of compression elements is spaced apart axially.

12. The outer sheath of claim 1, wherein the at least one compression element is a single compression element extending helically around the tubular member.

13. An outer sheath, comprising:
a tubular member defined by a circumferential wall and having a distal end, a proximal end, and a lumen extending therebetween along a central longitudinal axis of the tubular member, the tubular member having a distal region being configured to shift between an enlarged configuration and a collapsed configuration; and
at least one compression element embedded within the circumferential wall in at least the distal region, the at least one compression element configured to radially collapse the distal region when the lumen of the distal region is empty.

14. The outer sheath of claim 13, wherein the at least one compression element is configured to automatically collapse the distal region when the lumen is empty.

15. The outer sheath of claim 13, wherein the at least one compression element extends helically around the tubular member.

16. The outer sheath of claim 13, wherein the at least one compression element extends along an entirety of the distal region.

17. A medical device system, comprising:
a replacement heart valve delivery system; and
an outer sheath comprising:
a tubular member defined by a circumferential wall and having a distal end, a proximal end, and a lumen extending therebetween along a central longitudinal axis of the tubular member, the tubular member having a distal region being configured to shift between an enlarged configuration to a collapsed configuration; and
a plurality of compression elements disposed in the distal region, the plurality of compression elements configured to automatically radially collapse the distal region when the lumen of the distal region is empty;
wherein the replacement heart valve delivery system is configured to slide within the lumen of the tubular member.

18. The medical device system of claim 17, wherein a distal section of the replacement heart valve delivery system includes a replacement heart valve implant disposed therein, when in the enlarged configuration, the distal region of the tubular member is configured to extend over the distal section of the replacement heart valve delivery system containing the replacement heart valve implant.

19. The medical device system of claim 18, wherein a first outer diameter of the distal region of the tubular member when extended over the distal section of the replacement heart valve delivery system containing the replacement heart valve implant, is larger than a second outer diameter of a remainder of the outer sheath.

20. The medical device system of claim 19, wherein when the replacement heart valve delivery system is moved distally out of the outer sheath and the replacement heart valve implant is deployed, the plurality of compression elements is configured to radially collapse the distal region of the tubular member.
